(19) United States
(12) Patent Application Publication
(10) Pub. No.: US 2026/0184736 A1
(43) Pub. Date: Jul. 2, 2026

(54) METHOD OF SYNTHESIZING 4'-PHOSPHATE ANALOG NUCLEOTIDE PHOSPHORAMIDITE

(71) Applicant: ELI LILLY AND COMPANY, Indianapolis, IN (US)

(72) Inventors: Scott Alan FRANK, Indianapolis, IN (US); John Robert RIZZO, Indianapolis, IN (US)

(21) Appl. No.: 19/131,596

(22) PCT Filed: Nov. 22, 2023
(86) PCT No.: PCT/US2023/080878

§ 371 Date: May 21, 2025

Related U.S. Application Data
(60) Provisional application No. 63/427,488, filed on Nov. 23, 2022.

(51) Int. Cl. C07H 19/10; C07H 1/00

(52) U.S. Cl. CPC C07H 19/10; C07H 1/00

(57) ABSTRACT

Methods are disclosed for making a 4′-phosphate analog phosphoramidite known as MeMOP, which can be used in the synthesis of oligonucleotides such as therapeutic oligonucleotides.

### TECHNICAL FIELD

The disclosure relates generally to an improved method of making a nucleotide phosphoramidite, including 4′-phosphate analog such as 2-cyanoethyl ((2R,3S,4R,5R)-2-((dimethoxyphosphoryl)methoxy)-5-(2,4-dioxo-3,4-dihydropyrimidin-1 (2H)-yl)-4-methoxytetrahydrofuran-3-yl) diisopropylphosphoramidite (methoxy, phosphonate-4′-oxy-2′-O-methyluridine, MePhosphonate-40-mU or MeMOP), which can be used in making therapeutic oligonucleotides.

### BACKGROUND

Oligonucleotides are short, polymeric sequences of nucleotides that have a wide range of applications, including use as primers, probes and therapeutics. Oligonucleotides, such as therapeutic oligonucleotides, can be chemically synthesized using a variety of known methods. Various chemical modifications can be made to one or more of the nucleotides in therapeutic oligonucleotides to introduce improved properties for in vivo administration (e.g., to stabilize an oligonucleotide against nucleases, to increase cellular uptake of the oligonucleotide, and/or to enhance other pharmacodynamic and/or pharmacokinetic properties of the oligonucleotide).

Intl. Patent Application Publication No. WO 2018/045317 describes a method of making 4′-phosphate analog known as MeMOP to improve therapeutic oligonucleotides for in vivo administration. The method described therein uses a lead (Pb)-based reagent that is not available at scale, is highly toxic, and is environmentally hazardous.

In view of the above, there is a need for an improved method of making MeMOP.

### BRIEF SUMMARY

The disclosure describes a method of making MeMOP. In one instance, a method of making MeMOP is provided that includes the following steps:
(1) oxidizing 1-((2R,3R,4R,5R)-4-hydroxy-5-(hydroxymethyl)-3-methoxytetrahydrofuran-2-yl)pyrimidine-2,4 (1H,3H)-dione to obtain (2S,3S,4R,5R)-5-(2,4-dioxo-3,4-dihydropyrimidin-1 (2H)-yl)-3-hydroxy-4-methoxytetrahydrofuran-2-carboxylic acid;
(2) amidating and silylating (2S,3S,4R,5R)-5-(2,4-dioxo-3,4-dihydropyrimidin-1 (2H)-yl)-3-hydroxy-4-methoxytetrahydrofuran-2-carboxylic acid to obtain (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide;
(3) adding an organometallic moiety to (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide to obtain 1-((2R,3R,4S,5S)-5-acetyl-4-((tert-butyldimethylsilyl)oxy)-3-methoxytetrahydrofuran-2-yl)pyrimidine-2,4 (1H,3H)-dione;
(4) oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione to obtain (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate;
(5) desilylating and benzoylating (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate to obtain (2R,3S,4R,5R)-2-acetoxy-5-(2,4-dioxo-3,4-dihydropyrimidin-1 (2H)-yl)-4-methoxytetrahydrofuran-3-yl benzoate;
(6) hydrolyzing and alkylating (2R,3S,4R,5R)-2-acetoxy-5-(2,4-dioxo-3,4-dihydropyrimidin-1 (2H)-yl)-4-methoxytetrahydrofuran-3-yl benzoate to obtain [(2R,3S,4R,5R)-2-(dimethoxyphosphorylmethoxy)-5-(2,4-dioxopyrimidin-1-yl)-4-methoxy-tetrahydrofuran-3-yl]benzoate;
(7) debenzoylating [(2R,3S,4R,5R)-2-(dimethoxyphosphorylmethoxy)-5-(2,4-dioxopyrimidin-1-yl)-4-methoxy-tetrahydrofuran-3-yl]benzoate to obtain dimethyl ((((2R,3S,4R,5R)-5-(2,4-dioxo-3,4-dihydropyrimidin-1 (2H)-yl)-3-hydroxy-4-methoxytetrahydrofuran-2-yl)oxy)methyl)phosphonate; and
(8) phosphorylating dimethyl ((((2R,3S,4R,5R)-5-(2,4-dioxo-3,4-dihydropyrimidin-1 (2H)-yl)-3-hydroxy-4-methoxytetrahydrofuran-2-yl)oxy)methyl)phosphonate to obtain 2-cyanoethyl ((2R,3S,4R,5R)-2-((dimethoxyphosphoryl)methoxy)-5-(2,4-dioxo-3,4-dihydropyrimidin-1 (2H)-yl)-4-methoxytetrahydrofuran-3-yl) diisopropylphosphoramidite (MeMOP).

In some instances, a compound represented by a structure of:

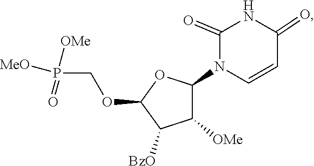

can be prepared by a method that includes the following steps:
(1) oxidizing 1-((2R,3R,4R,5R)-4-hydroxy-5-(hydroxymethyl)-3-methoxytetrahydrofuran-2-yl)pyrimidine-2,4 (1H,3H)-dione to obtain (2S,3S,4R,5R)-5-(2,4-dioxo-3,4-dihydropyrimidin-1 (2H)-yl)-3-hydroxy-4-methoxytetrahydrofuran-2-carboxylic acid;
(2) amidating and silylating (2S,3S,4R,5R)-5-(2,4-dioxo-3,4-dihydropyrimidin-1 (2H)-yl)-3-hydroxy-4-methoxytetrahydrofuran-2-carboxylic acid to obtain (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide;
(3) adding an organometallic moiety to (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide to obtain 1-((2R,3R,4S,5S)-5-acetyl-4-((tert-butyldimethylsilyl)oxy)-3-methoxytetrahydrofuran-2-yl)pyrimidine-2,4 (1H,3H)-dione;
(4) oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione to obtain (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate;
(5) desilylating and benzoylating (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate to obtain (2R,3S,4R,5R)-2-acetoxy-5-(2,4-dioxo-3,4-dihydropyrimidin-1 (2H)-yl)-4-methoxytetrahydrofuran-3-yl benzoate; and
(6) hydrolyzing and alkylating (2R,3S,4R,5R)-2-acetoxy-5-(2,4-dioxo-3,4-dihydropyrimidin-1 (2H)-yl)-4-methoxytetrahydrofuran-3-yl benzoate to obtain [(2R,3S,4R,5R)-2-(dimethoxyphosphorylmethoxy)-5-(2,4-dioxopyrimidin-1-yl)-4-methoxy-tetrahydrofuran-3-yl]benzoate.

In some instances, the step of oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione can include a Baeyer Villiger reaction. In some instances, the Baeyer Villiger reaction includes the use of meta-chloroperoxybenzoic acid (mCPBA) or urea hydrogen peroxide (UHP).

An advantage of the methods herein is that the materials used in the method are available at commercial scale.

An advantage of the methods herein is that the materials used therein are non-toxic.

An advantage of the methods herein is that the materials used therein are not environmentally hazardous.

An advantage of the methods herein is that they are more cost effective and provide MeMOP in higher yields and purity as compared to known methods of making MeMOP.

An advantage of the methods herein is that they use a Baeyer Villiger reaction for making MeMOP, which is a stereo-specific process that gives a desired β-anomer (vs α-anomer) exclusively in the 4′ OH position of the ribose.

### BRIEF DESCRIPTION OF THE DRAWINGS

The advantages, effects, features, and objects other than those set forth above will become more readily apparent when consideration is given to the detailed description below. Such detailed description refers to the following drawing(s), where:

FIG. 1A and FIG. 1B depict an exemplary scheme for making MeMOP.

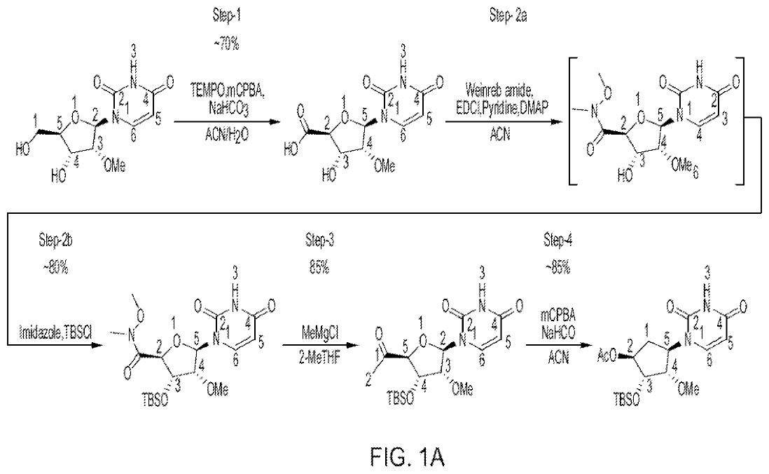

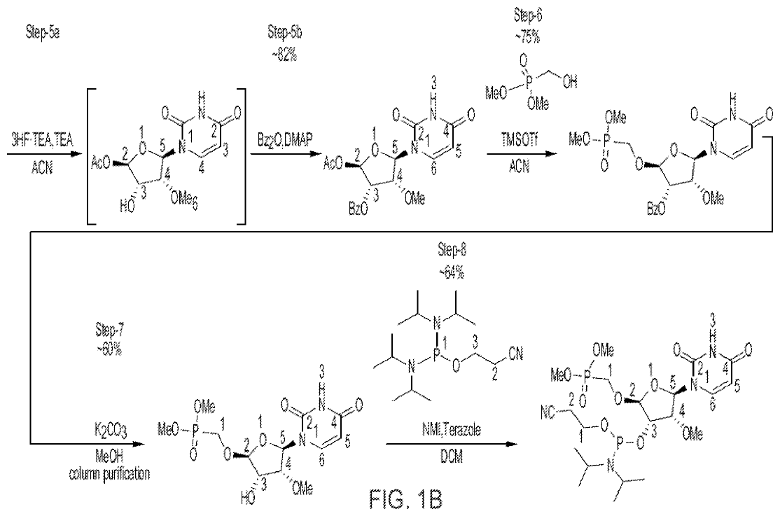

### DETAILED DESCRIPTION

### Overview

Chemical modifications can be introduced into a therapeutic oligonucleotide to confer properties that may be desired under specific conditions, such as conditions experienced following its in vivo administration. These modifications can be introduced in the base, sugar, and/or phosphate group of one or more nucleotides of the oligonucleotide. Such modifications include those designed, for example: (i) to stabilize the oligonucleotide against nucleases or other enzymes that degrade or interfere with the structure or activity of the oligonucleotide, (ii) to increase cellular uptake of the oligonucleotide, and/or (iii) to improve the pharmacokinetic properties of the oligonucleotide.

For example, a therapeutic oligonucleotide can include a hydroxyl group at a 5′-terminus or a 3′-terminus. It is possible to replace the hydroxyl group with a phosphate group, for example, to attach linkers, adapters, labels and/or targeting ligands, or to directly ligate the oligonucleotide to another nucleic acid. In addition, the phosphate group can enhance the interaction between the oligonucleotide and a protein such as, for example, Argonaute 2 (Ago2). However, a phosphate group at the 5′-terminus can be susceptible to degradation via phosphatases or other enzymes, which can limit their in vivo bioavailability. As such, phosphate analogs have been developed that can be incorporated into a therapeutic oligonucleotide that not only provide a functional effect of a phosphate group but also are more stable in vivo. One such phosphate analog is MeMOP, which is a 4′-phosphate analog nucleotide phosphoramidite. See, Intl. Patent Application Publication No. WO 2018/045317.

### Abbreviations and Definitions

Unless defined otherwise, all technical and scientific terms used herein have the same meaning as commonly understood by one of skill in the art to which the disclosure pertains. Although any methods and materials similar to or equivalent to those described herein can be used in the practice or testing of the methods, the exemplary methods and materials are described herein.

Additionally, reference to an element by the indefinite article “a” or “an” does not exclude the possibility that more than one element is present, unless the context clearly requires that there be one and only one element. The indefinite article “a” or “an” thus usually means “at least one.”

Moreover, use of “including,” as well as other forms, such as “include,” “includes” and “included” is not limiting.

Certain abbreviations used herein are as follows:
“ACN” refers to acetonitrile (C₂H₃N); “DCM” refers to dichloromethane (CH₂Cl₂); “DMAP” refers to 4-dimethylaminopyridine (C₇H₁₀N₂); “DMSO” refers to dimethyl sulfoxide (C₂H₆OS); “DMHMP” refers to dimethyl P-(hydroxymethyl)phosphonate (C₃H₉O₄P); “DNA” refers to deoxyribonucleic acid; “EDCI” refers to 1-ethyl-3-(3-dimethylaminopropyl) carbodiimide (C₈H₁₇N₃); “ES-MS” refers to electrospray mass spectrometry; “EtOAc” refers to ethyl acetate (C₄H₈O₂); “eq” refers to equivalent(s); “hr” refers to hour(s); “mCPBA” refers to meta-chloroperoxybenzoic acid (C₇H₅ClO₃); “Me” refers to methyl (—CH₃); “MeOH” refers to methanol (CH₄O); “min” refers to minute(s); “MTBE” refers to methyl tertiary-butyl ether (C₅H₁₂O); “′m/z” refers to mass-to-charge ratio; “NMI” refers to N-methylimidazole (C₄H₆N₂); “NODMHA*HCl” refers to N,O-dimethylhydroxylamine hydrochloride (C₂H₈ClNO or C₂H₇NO*HCl); “RNA” refers to ribonucleic acid; “TBSCl” refers to tert-butyldimethylsilyl chloride (C₆H₁₅ClSi); “TBSO” refers to tert-butyldimethylsilyl ether; “TEA” refers to triethylamine (C₆H₁₅N); “TEMPO” refers to 2,2,6,6-tetramethyl-1-piperidinyloxy (C₉H₁₈NO); “THF” refers to tetrahydrofuran (C₄H₈O); “TMSOTf” refers to trimethylsilyl trifluoromethanesulfonate (C₄H₉F₃O₃SSi); “UHP” refers to urea hydrogen peroxide; “V” refers to volume(s).

Certain definitions used herein are as follows:

As used herein, “about” means within a statistically meaningful range of a value or values such as, for example, a stated concentration, length, molecular weight, pH, sequence similarity, time frame, temperature, volume, etc. Such a value or range can be within 20%, within 15%, within 10%, or more typically within 5% of a given value or range. Alternatively, and with respect to biological systems or processes “about” can mean within an order of magnitude such as, for example, within five-fold or more typically within two-fold of a given value. The allowable variation encompassed by “about” will depend upon the system under study, and can be readily appreciated by one of skill in the art.

As used herein, “modified nucleobase” means a nucleobase including a modified purine or pyrimidine base (e.g., adenine (A), guanine (G), cytosine (C), thymine (T) and uracil (U)). Examples of modified nucleobases include, but are not limited to, diaminopurine and its derivatives, alkylated purines or pyrimidines, acylated purines or pyrimidines thiolated purines or pyrimidines, and the like. Other modified nucleobases include analogs of purines and pyrimidines including, but not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methycytosine, 5-methycytosine, 5-ethycytosine, 4-acetycytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl) uracil, 5-(methylaminomethyl) uracil, 5-(carboxymethylaminomethyl)-uracil, 2-thiouracil, 5-methyl-2-thiouracil, 5-(2-bromovinyl) uracil, uracil-5-oxyacetic acid, uracil-5-oxyacetic acid methylester, pseudouracil, 1-methylpseudouracil, queosine, hypoxanthine, xanthine, 2-aminopurine, 6-hydroxyarninopurine, nitropyrrolyl, nitroindolyl and difluorotolyl, 6-thiopurine and 2,6-diaminopurine nitropyrrolyl, nitroindolyl and difluorotolyl. Alternatively, a modified nucleobase may not contain a nitrogen atom (i.e., a universal base). See also, Intl. Patent Application Publication No. WO 2003/040395. Alternatively, the modified nucleobase is abasic (i.e., does not include a nucleobase).

As used herein, “modified nucleoside” means a nucleoside including a modified or universal nucleobase and/or a modified sugar. The modified or universal nucleobase (also referred to herein as a base analog) can be located at the 1′-position of the sugar moiety and refer to nucleobases other than adenine (A), guanine (G), cytosine (C), thymine (T) and uracil (U) at the 1′-position. In some instances, the modified nucleotide does not contain a nucleobase (abasic). The modified sugar (also referred to herein as a sugar analog) includes modified deoxyribose or ribose moieties (e.g., where the modification occurs at the 2′-, 3′-, 4′- or 5′-carbon position of the sugar). The modified sugar may also include non-natural alternative carbon structures such as those present in bridged nucleic acids (“BNA”), locked nucleic acids (“LNA”) and/or unlocked nucleic acid (“UNA”).

As used herein, “modified nucleotide” means a nucleotide including a modified or universal nucleobase as described above, a modified sugar as described above, and/or a modified phosphate or phosphate group. The modified phosphate can be a modification of the phosphate or phosphate group that does not occur in natural nucleotides and includes non-naturally occurring phosphate mimics as are known in the art. Modified phosphate or phosphate groups also include non-naturally occurring internucleotide linking groups, including both phosphorous-containing linking groups and non-phosphorous-containing linking groups as are known in the art. Suitable modified or universal nucleobases, modified sugars, and modified phosphates or phosphate groups are described herein.

As used herein, “nucleobase” means a heterocyclic nitrogenous base capable of forming Watson-Crick-type hydrogen bonds and stacking interactions in pairing with a complementary nucleobase or nucleobase analog (i.e., derivatives of nucleobases) when that nucleobase is incorporated into a polymeric structure. The natural heterocyclic nitrogenous bases include purines and pyrimidines such as adenine (A), guanine (G), cytosine (C), thymine (T) and uracil (U).

As used herein, “nucleoside” means a heterocyclic nitrogenous base in N-glycosidic linkage with a sugar moiety (e.g., deoxyribose, ribose or analog thereof).

As used herein, “nucleoside phosphoramidite” means a derivative of a natural or synthetic nucleoside in which reactive hydroxy and exocyclic amino groups present in natural or synthetic nucleosides are appropriately protected to prevent undesired side reactions during the synthesis of nucleic acids.

As used herein, “nucleotide” means a heterocyclic nitrogenous base in N-glycosidic linkage with a sugar moiety (e.g., deoxyribose, ribose or analog thereof) that is linked to a phosphate or phosphate group (i.e., nucleoside plus phosphate or phosphate group). As above, natural heterocyclic nitrogenous bases include adenine (A), guanine (G), cytosine (C), thymine (T) and uracil (U).

As used herein, “nucleotide phosphoramidite” means a derivative of a natural or synthetic nucleotide in which reactive hydroxy and exocyclic amino groups present in natural or synthetic nucleotides are appropriately protected to prevent undesired side reactions during nucleic acid synthesis.

As used herein, “oligonucleotide” means a short nucleic acid (e.g., less than about 100 nucleotides in length) of ribonucleotides, deoxyribonucleotides or a combination thereof. An oligonucleotide may be single-stranded (ss) or double-stranded (ds). An oligonucleotide may or may not have duplex regions. As a set of non-limiting examples, the oligonucleotide may be, but is not limited to, a small interfering RNA (siRNA), microRNA (miRNA), short hairpin RNA (shRNA), dicer substrate interfering RNA (dsiRNA), antisense oligonucleotide (ASO), short siRNA or ss siRNA.

As used herein, “phosphate analog” means a chemical moiety that mimics the electrostatic and/or steric properties of a phosphate group. A phosphate analog can be positioned at the 5′ terminal nucleotide of an oligonucleotide in place of a 5′-phosphate, which can include a phosphatase-resistant linkage. Examples of phosphate analogs include, but are not limited to, 5′ phosphonates, such as 5′ methylene phosphonate (5′-MP) and 5′-(E)-vinylphosphonate (5′-VP). Likewise, a phosphate analog can be positioned at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., Intl. Patent Application Publication No. WO 2018/045317. Other modifications have been developed for the 5′ end of oligonucleotides (see, e.g., Intl. Patent Application No. WO 2011/133871; U.S. Pat. No. 8,927,513; and Prakash et al. (2015) Nuc. Acids Res. 43:2993-3011).

As used herein, “phosphoramidite” means a nitrogen-containing, trivalent phosphorus derivative that can have a formula of (RO)₂PNR₂.

As used herein, “protecting group” means a group that reversibly renders unreactive a functional group under certain conditions of a desired reaction. After the desired reaction, the protecting group can be removed to deprotect the protected functional group. The protecting group should be removable under conditions that do not degrade a substantial proportion of the molecule (i.e., an oligonucleotide) being synthesized.

As used herein, “ribonucleotide” means a natural or modified nucleotide that has a hydroxyl group at the 2′-position of the sugar moiety.

As used herein, “targeting ligand” means a chemical moiety that facilitates entry of an oligonucleotide such as an RNAi agent into a cell. It can be a compound (e.g., an amino sugar, carbohydrate, cholesterol, lipid or polypeptide) that selectively binds to a cognate compound (e.g., a receptor) of a tissue or cell of interest and that is conjugatable to another substance for targeting another substance to the tissue or cell of interest. For example, a targeting ligand may be conjugated to an oligonucleotide for purposes of targeting it to a specific tissue or cell of interest. A targeting ligand can selectively bind to a cell surface receptor. Accordingly, a targeting ligand, when conjugated to an oligonucleotide, facilitates delivery of the oligonucleotide into a particular cell through selective binding to a receptor expressed on the surface of the cell and endosomal internalization by the cell of the complex comprising the oligonucleotide, targeting ligand, and receptor. Moreover, a targeting ligand can be conjugated to an oligonucleotide via a linker that is cleaved following or during cellular internalization such that the oligonucleotide is released from the targeting ligand in the cell.

### Compositions

### MeMOP:

The structure of MeMOP is as follows:

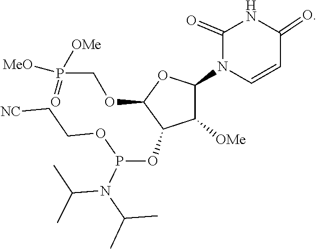 [SMILES: CO[C@@H]1[C@H](OP(OCCC#N)N(C(C)C)C(C)C)[C@@H](OCP(=O)(OC)OC)O[C@H]1n1ccc(=O)[nH]c1=O]

See, Intl. Patent Application Publication No. WO 2018/045317.

### MeMOP-Modified Oligonucleotides:

MeMOP can be incorporated into an oligonucleotide, such as a therapeutic oligonucleotide. In some instances, MeMOP can be bound to a 4′-carbon of a sugar moiety (e.g., a ribose, a deoxyribose or an analog thereof) of a nucleotide within the oligonucleotide. In some instances, MeMOP can be incorporated at the 3′-terminus of the oligonucleotide. In other instances, MeMOP can be incorporated at the 5′-terminus of the oligonucleotide. In yet other instances, MeMOP can be incorporated at both of the 5′-terminus and 3′ terminus of the oligonucleotide. In yet other instances, MeMOP can be incorporated at one or more internal positions of the oligonucleotide. See, e.g., Intl. Patent Application Publication Nos. WO 2018/045317, WO 2021/188795, WO 2022/032288 and WO 2022/221430.

Oligonucleotides (e.g., a ds oligonucleotide such as a MeMOP-modified oligonucleotide) can be made using methods and/or techniques known to one of skill in the art such as, for example, conventional nucleic acid solid-phase synthesis. The nucleotides of the oligonucleotides can be assembled on a suitable nucleic acid synthesizer utilizing standard nucleotide or nucleoside precursors (e.g., phosphoramidites). Automated nucleic acid synthesizers, including DNA/RNA synthesizers, are commercially available from, for example, Applied Biosystems (Foster City, CA), BioAutomation (Irving, TX) and GE Healthcare Life Sciences (Pittsburgh, PA).

In some instances, oligonucleotide synthesis steps can be performed in an alternate order to give the desired compounds. Other synthetic chemistry transformations, protecting groups (e.g., for hydroxyl, amino, etc. present on the bases), and protecting group methodologies (protection and deprotection) useful in synthesizing the oligonucleotides are known in the art and are described in, for example, Larock, “Comprehensive Organic Transformations,” VCH Publishers (1989); Greene & Wuts, “Protective Groups in Organic Synthesis,” 2nd Ed., John Wiley & Sons (1991); Fieser & Fieser, “Fieser & Fieser's Reagents for Organic Synthesis,” John Wiley & Sons (1994); and Paquette, ed., “Encyclopedia of Reagents for Organic Synthesis,” John Wiley & Sons (1995).

### Pharmaceutical Compositions:

MeMOP-modified oligonucleotides (or a pharmaceutically acceptable salt thereof such as, for example, trifluroacetate salts, acetate salts or hydrochloride salts) can be incorporated into a pharmaceutical composition, which includes an effective amount of MeMOP-containing oligonucleotides and a pharmaceutically acceptable carrier, delivery agent or excipient. See, e.g., Intl. Patent Application Publication Nos. WO 2018/045317, WO 2021/188795, WO 2022/032288 and WO 2022/221430.

Various formulations have been developed to facilitate oligonucleotide use. In some instances, oligonucleotides can be delivered to an individual or a cellular environment using a formulation that minimizes degradation, facilitates delivery and/or uptake, or provides another beneficial property to the oligonucleotides in the formulation. In some instances, the oligonucleotides can be formulated in buffer solutions such as phosphate buffered saline solutions, liposomes, micellar structures and capsids.

In some instances, oligonucleotides can be reacted with an inorganic and organic acid/base to form pharmaceutically acceptable acid/base addition salts. In some instances, forming a pharmaceutically acceptable acid/base addition salt improves the in vivo compatibility and/or effectiveness of the oligonucleotide. Pharmaceutically acceptable salts and common methodologies for preparing them are well known in the art (see, e.g., Stahl et al., “Handbook of Pharmaceutical Salts: Properties, Selection and Use,” 2nd Revised Edition (Wiley-VCH, 2011)). Pharmaceutically acceptable salts for use herein include sodium, trifluoroacetate, hydrochloride, and acetate salts.

In some instances, pharmaceutical compositions can be formulated to be compatible with an intended route of administration. Routes of administration include, but are not limited to, parenteral (e.g., intravenous, intramuscular, intraperitoneal, intradermal, and subcutaneous), oral (e.g., inhalation), transdermal (e.g., topical), transmucosal and rectal administration.

Moreover, factors such as solubility, bioavailability, biological half-life, route of administration, product shelf life, as well as other pharmacological considerations can be contemplated by one of skill in the art of preparing such pharmaceutical formulations, and as such, a variety of dosages and treatment regimens may be desirable.

In some instances, the pharmaceutical composition can include one or more additional therapeutic agents.

### Methods

### Method of Making MeMOP:

The methods of making MeMOP or a salt thereof can include the steps described herein, which may be, but not necessarily, carried out in the sequence as described. Other sequences, however, also are conceivable. Furthermore, individual or multiple steps may be carried out either in parallel and/or overlapping in time and/or individually or in multiply repeated steps. The products of each step below can be recovered by conventional methods, including chromatography, crystallization, evaporation, extraction, filtration, precipitation and trituration.

In one instance, MeMOP can be prepared according to the method below, which can include the following steps:
(1) oxidizing 1-((2R,3R,4R,5R)-4-hydroxy-5-(hydroxymethyl)-3-methoxytetrahydrofuran-2-yl)pyrimidine-2,4 (1H,3H)-dione

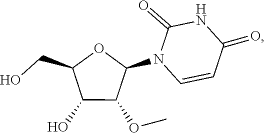 [SMILES: CO[C@@H]1[C@H](O)[C@@H](CO)O[C@H]1n1ccc(=O)[nH]c1=O]

to obtain (2S,3S,4R,5R)-5-(2,4-dioxo-3,4-dihydropyrimidin-1 (2H)-yl)-3-hydroxy-4-methoxytetrahydrofuran-2-carboxylic acid

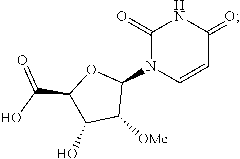 [SMILES: CO[C@@H]1[C@H](O)[C@@H](C(=O)O)O[C@H]1n1ccc(=O)[nH]c1=O]

(2) amidating and silylating (2S,3S,4R,5R)-5-(2,4-dioxo-3,4-dihydropyrimidin-1 (2H)-yl)-3-hydroxy-4-methoxytetrahydrofuran-2-carboxylic acid to obtain (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide

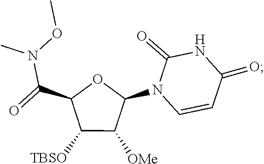 [SMILES: CO[C@@H]1[C@H](O[Si](C)(C)C(C)(C)C)[C@@H](C(=O)N(C)OC)O[C@H]1n1ccc(=O)[nH]c1=O]

(3) adding an organometallic moiety (e.g., an organomagnesium compound) to (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide to obtain 1-((2R,3R,4S,5S)-5-acetyl-4-((tert-butyldimethylsilyl)oxy)-3-methoxytetrahydrofuran-2-yl)pyrimidine-2,4 (1H,3H)-dione,

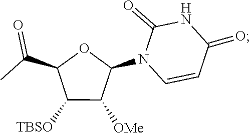 [SMILES: CO[C@@H]1[C@H](O[Si](C)(C)C(C)(C)C)[C@@H](C(C)=O)O[C@H]1n1ccc(=O)[nH]c1=O]

(4) oxidizing (e.g., via a Baeyer-Villiger reaction) 1-((2R,3R,4S,5S)-5-acetyl-4-((tert-butyldimethylsilyl)oxy)-3-methoxytetrahydrofuran-2-yl)pyrimidine-2,4 (1H,3H)-dione to obtain (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate

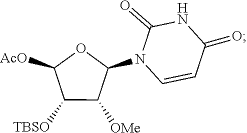 [SMILES: CO[C@@H]1[C@H](O[Si](C)(C)C(C)(C)C)[C@@H](OC(C)=O)O[C@H]1n1ccc(=O)[nH]c1=O]

(5) desilylating and benzoylating (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate to obtain (2R,3S,4R,5R)-2-acetoxy-5-(2,4-dioxo-3,4-dihydropyrimidin-1 (2H)-yl)-4-methoxytetrahydrofuran-3-yl benzoate

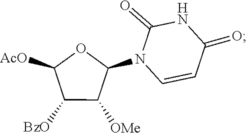

(6) hydrolyzing and alkylating (2R,3S,4R,5R)-2-acetoxy-5-(2,4-dioxo-3,4-dihydropyrimidin-1 (2H)-yl)-4-methoxytetrahydrofuran-3-yl benzoate to obtain [(2R,3S,4R,5R)-2-(dimethoxyphosphorylmethoxy)-5-(2,4-dioxopyrimidin-1-yl)-4-methoxy-tetrahydrofuran-3-yl]benzoate

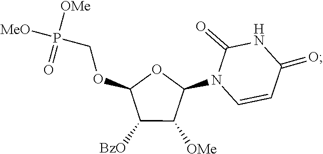

(7) debenzoylating [(2R,3S,4R,5R)-2-(dimethoxyphosphorylmethoxy)-5-(2,4-dioxopyrimidin-1-yl)-4-methoxy-tetrahydrofuran-3-yl]benzoate to obtain dimethyl ((((2R,3S,4R,5R)-5-(2,4-dioxo-3,4-dihydropyrimidin-1 (2H)-yl)-3-hydroxy-4-methoxytetrahydrofuran-2-yl)oxy)methyl)phosphonate,

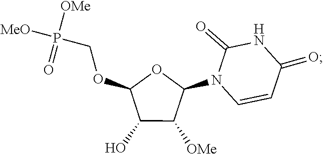 [SMILES: CO[C@@H]1[C@H](O)[C@@H](OCP(=O)(OC)OC)O[C@H]1n1ccc(=O)[nH]c1=O]

and
(8) phosphorylating dimethyl(2R,3S,4R,5R)-2-acetoxy-5-(2,4-dioxo-3,4-dihydropyrimidin-1 (2H)-yl)-4-methoxytetrahydrofuran-3-yl benzoate to obtain 2-cyanoethyl ((2R,3S,4R,5R)-2-((dimethoxyphosphoryl)methoxy)-5-(2,4-dioxo-3,4-dihydropyrimidin-1 (2H)-yl)-4-methoxytetrahydrofuran-3-yl) diisopropylphosphoramidite (MeMOP)

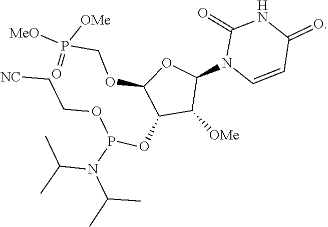 [SMILES: CO[C@@H]1[C@H](OP(OCCC#N)N(C(C)C)C(C)C)[C@@H](OCP(=O)(OC)OC)O[C@H]1n1ccc(=O)[nH]c1=O]

In some instances, [(2R,3S,4R,5R)-2-(dimethoxyphosphorylmethoxy)-5-(2,4-dioxopyrimidin-1-yl)-4-methoxy-tetrahydrofuran-3-yl]benzoate

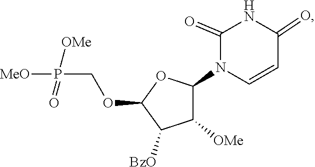

can be prepared according to the method below, which can include the following steps:
(1) oxidizing 1-((2R,3R,4R,5R)-4-hydroxy-5-(hydroxymethyl)-3-methoxytetrahydrofuran-2-yl)pyrimidine-2,4 (1H,3H)-dione to obtain (2S,3S,4R,5R)-5-(2,4-dioxo-3,4-dihydropyrimidin-1 (2H)-yl)-3-hydroxy-4-methoxytetrahydrofuran-2-carboxylic acid;
(2) amidating and silylating (2S,3S,4R,5R)-5-(2,4-dioxo-3,4-dihydropyrimidin-1 (2H)-yl)-3-hydroxy-4-methoxytetrahydrofuran-2-carboxylic acid to obtain (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide;
(3) adding an organometallic moiety to (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide to obtain 1-((2R,3R,4S,5S)-5-acetyl-4-((tert-butyldimethylsilyl)oxy)-3-methoxytetrahydrofuran-2-yl)pyrimidine-2,4 (1H,3H)-dione;
(4) oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione to obtain (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate;
(5) desilylating and benzoylating (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate to obtain (2R,3S,4R,5R)-2-acetoxy-5-(2,4-dioxo-3,4-dihydropyrimidin-1 (2H)-yl)-4-methoxytetrahydrofuran-3-yl benzoate; and
(6) hydrolyzing and alkylating (2R,3S,4R,5R)-2-acetoxy-5-(2,4-dioxo-3,4-dihydropyrimidin-1 (2H)-yl)-4-methoxytetrahydrofuran-3-yl benzoate to obtain [(2R,3S,4R,5R)-2-(dimethoxyphosphorylmethoxy)-5-(2,4-dioxopyrimidin-1-yl)-4-methoxy-tetrahydrofuran-3-yl]benzoate.

In some instances, the step of oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione can be a Baeyer Villiger reaction, which is a stereo-specific process that gives the desired β-anomer exclusively in the 4′OH position of the ribose. In some instances, the Baeyer Villiger reaction can be carried out with meta-chloroperoxybenzoic acid (mCPBA) or urea hydrogen peroxide (UHP).

### Examples

The following non-limiting example(s) are offered for purposes of illustration, and are not limiting to the scope of the disclosure.

### Example 1: Synthesizing (2S,3S,4R,5R)-5-(2,4-dioxo-3,4-dihydropyrimidin-1 (2H)-yl)-3-hydroxy-4-methoxytetrahydrofuran-2-carboxylic acid

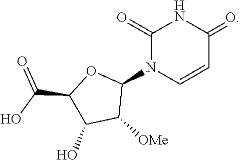 [SMILES: CO[C@@H]1[C@H](O)[C@@H](C(=O)O)O[C@H]1n1ccc(=O)[nH]c1=O]

Method: 1-((2R,3R,4R,5R)-4-hydroxy-5-(hydroxymethyl)-3-methoxytetrahydrofuran-2-yl)pyrimidine-2,4 (1H,3H)-dione (1.0 eq) was added to a solution of ACN (5 V), H₂O (5 V), TEMPO (0.5 eq), and NaHCO₃ (4.0 eq) and mCPBA (4.0 eq) slowly in portions at 15° C. to 25° C. The resulting mixture was warmed to 30° C. to 40° C. and was stirred for 4 hr at room temperature. The mixture was cooled, and the solution was quenched by adding 20% aqueous NaHSO₃. The mixture was then diluted with EtOAc (10 V), and the pH was adjusted to 1˜2 with 36% aqueous HCl. The mixture was filtered to obtain a first wet cake. The first filtrate was collected, and the organic phase was removed. The aqueous phase was concentrated to 3 V to form a suspension. The suspension was filtered, and a second wet cake was obtained. The first and second wet cakes were combined and successively washed with EtOAc (4 V) and water (1 V). The solid was dried under vacuum to afford the title compound (70%) as a solid.

Result: ¹H-NMR (DMSO-d₆) δ 3.32 (s, 3H, OCH₃), 3.83 (q, 1H, C₄′H,),4.35 (s, 2H, C2′H, C3′H), 5.76 (q, 1H, C5-H), 5.84 (br, 1H, C3′OH), 6.05 (d, 1H, C1′H), 8.14 (d, 1H, C6-H), 11.40 (s, 1H, NH). ES-MS m/z 271.00 (M−H).

### Example 2: Synthesizing (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide

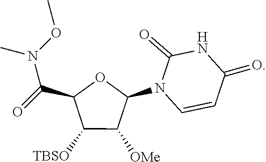 [SMILES: CO[C@@H]1[C@H](O[Si](C)(C)C(C)(C)C)[C@@H](C(=O)N(C)OC)O[C@H]1n1ccc(=O)[nH]c1=O]

Method: (2S,3S,4R,5R)-5-(2,4-dioxo-3,4-dihydropyrimidin-1 (2H)-yl)-3-hydroxy-4-methoxytetrahydrofuran-2-carboxylic acid (1.0 eq) of Example 1 was dissolved in a mixture of ACN (10 V), DMAP (0.13 eq) and pyridine (3.3 eq). EDCI (1.6 eq) was added slowly in portions while maintaining the temperature at −5° C. to 5° C. N,O-NODMHA*HCl (1.5 eq) is added slowly in portions while maintaining the temperature at −5° C. to 5° C. The solution was warmed to 10° C. to 20° C. and stirred for 2 hr. Imidazole (3.0 eq) and TBSCI (2.5 eq) were added and stirred for 16 hr at 10° C. to 20° C. The mixture was quenched by adding H₂O (2 V) and concentrated (3 V). The mixture was extracted with EtOAc (10 V) and then successively washed with 1 M aqueous HCl (5V×2), sat. aqueous NaHCO₃ (5 V), and 8% aqueous Na₂SO₄ (5 V). The mixture was concentrated to 4 V and n-heptane (10 V) was added. The mixture was then concentrated to 10 V, and the mixture was crystallized. The mixture was filtered and then dried to afford the title compound (80%) as a solid.

Result: ¹H NMR (DMSO-d₆) δ 0.11 (d, 6H, SiCH₃), 0.988 (s, 9H, SiCCH₃,), 3.17 (s, 3H, NCH₃), 3.29 (s, 3H, C2′OCH₃), 3.71 (s, 3H, NOCH₃), 3.85 (t, 1H, C4′H), 4.47 (dd, 1H, C3′H), 4.76 (s, 1H, C2′H), 5.76 (d, 1H, C5-H), 6.06 (d, 1H, C1′H), 8.50 (d, 1H, C6-H), 11.39 (s, 1H, CONH). ES-MS m/z 430.30 (M+H).

### Example 3: Synthesizing 1-((2R,3R,4S,5S)-5-acetyl-4-((tert-butyldimethylsilyl)oxy)-3-methoxytetrahydrofuran-2-yl)pyrimidine-2,4 (1H,3H)-dione

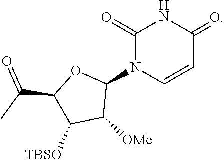 [SMILES: CO[C@@H]1[C@H](O[Si](C)(C)C(C)(C)C)[C@@H](C(C)=O)O[C@H]1n1ccc(=O)[nH]c1=O]

Method: (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide (1.0 eq) of Example 2 was dissolved in 2-MeTHF (15 V). A solution of 3 N MeMgCl in THF (2.3 eq) was added dropwise while maintaining the temperature at −20° C. to 10° C. The resulting mixture was stirred for 16 hr at −20° C. to 10° C. H₂O (1 V) was added, and the pH was adjusted to 4˜5 with 1 M aqueous HCl. The two phases are separated, and the organic phase was successively washed with sat. aqueous NaHCO₃ (5 V×2) and 8% aqueous Na₂SO₄ (5 V). The mixture was concentrated to 3 V, and n-heptane (10 V) was added to crystallize the mixture. The mixture was filtered, and the filter cake was dried to give the title compound (85%) as a solid.

Result: Weight: ¹H NMR (CDCl₃) δ 0.14 (s, 6H, SiCH₃), 0.93 (s, 9H, SiCCH₃,), 2.28 (s,3H, COCH₃), 3.49 (s, 3H, C2′OCH₃), 3.72 (t, 1H, C4′H), 4.14 (dd, 1H, C3′H), 4.64 (d, 1H, C2′H), 5.80 (d, 1H, C5-H), 5.94 (d, 1H, C1′H), 8.11 (d, 1H, C6-H), 8.60 (s, 1H, CONH). ES-MS m/z 385.20 (M+H).

### Example 4: Synthesizing (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate

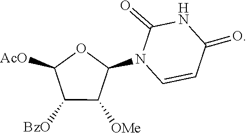

Method: 1-((2R,3R,4S,5S)-5-acetyl-4-((tert-butyldimethylsilyl)oxy)-3-methoxytetrahydrofuran-2-yl)pyrimidine-2,4 (1H,3H)-dione (1.0 eq) of Example 3 was dissolved in ACN (10 V). A solution of NaHCO₃ (3.0 eq) was added, followed by a slow addition of mCPBA (1.5 eq) at 5° C. to 15° C. The mixture was stirred for 3 hr at room temperature, then diluted with water (6 V) and quench with 20% aqueous NaHSO₃. The solution was extracted with EtOAc (10 V), and the layers were separated. The organic phase was successively washed with sat. aqueous NaHCO₃ (5 V×2) and 8% aqueous Na₂SO₄ (5 V). The mixture was concentrated to 3 V, and n-heptane (14 V) was added. The mixture was filtered, and the filter cake was dried to give the title compound (85%) as a solid.

Result: ¹H NMR (CDCl₃) δ 0.15 (s, 6H, SiCH₃), 0.93 (s, 9H, SiCCH₃,),2.17 (s,3H, COCH₃), 3.41 (s, 3H, C2′OCH₃), 3.90 (dd, 1H, C3′H), 4.26 (d, 1H, C2′H), 5.80 (d, 1H, C5-H), 6.03 (s, 1H, C4′H), 6.26 (d, 1H, C1′H), 7.39 (d, 1H, C6-H), 8.28 (br, 1H, CONH). ES-MS m/z 341.20 (M+H).

### Example 5: Synthesizing (2R,3S,4R,5R)-2-acetoxy-5-(2,4-dioxo-3,4-dihydropyrimidin-1 (2H)-yl)-4-methoxytetrahydrofuran-3-yl benzoate

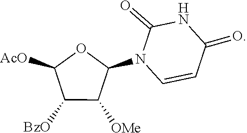

Method: (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate (1.0 eq) of Example 4 was dissolved in ACN (10 V), TEA (6.0 eq) and 3HF·TEA (3.0 eq). The mixture was warmed to 25° C. to 35° C. and then stirred for 6 hr. DMAP (0.1 eq) and Bz₂O(1.5 eq) were added, and the mixture was stirred for 2 hr at 20° C. to 30° C. H₂O (2 V) was added, and the mixture was concentrated to 4 V under vacuum. The mixture was extracted with EtOAc (8 V), and the layers were separated. The organic phase was washed successively with sat. aqueous NaHCO₃ (5 V), 1 M aqueous HCl (5 V) and 8% aqueous Na₂SO₄ (5 V). The mixture was concentrated to 2 V, and EtOAc (5 V) was added. The mixture was concentrated to 2 V, and n-heptane (10 V) was added. The suspension was stirred at 0° C. to 10° C. and then filtered. The filter cake was dried to give the title compound (82%) as solid.

Result: ¹H NMR (CDCl₃) δ 2.21 (s, 3H, COCH₃), 3.44 (s, 3H, C2′OCH₃), 4.26 (d, 1H, C2′H), 5.60 (d, 1H, C3′H), 5.85 (dd, 1H, C5-H), 6.36 (d, 1H, C1′H), 6.38 (s, 1H, C4′H), 7.37-7.65 (m, 6H, C6-H, Ph-H), 8.17 (s, 1H, CONH). ES-MS m/z 389.10 (M−H).

### Example 6: Synthesizing [(2R,3S,4R,5R)-2-(dimethoxyphosphorylmethoxy)-5-(2,4-dioxopyrimidin-1-yl)-4-methoxy-tetrahydrofuran-3-yl]benzoate

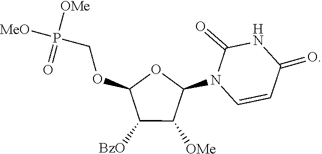

Method: (2R,3S,4R,5R)-2-acetoxy-5-(2,4-dioxo-3,4-dihydropyrimidin-1 (2H)-yl)-4-methoxytetrahydrofuran-3-yl benzoate (1.0 eq) of Example 5 was dissolved in ACN (5 V). TMSOTf (2.4 eq) was added dropwise while maintaining the temperature at −15° C. to −5° C. DMHMP (5.0 eq) was added dropwise to the mixture while maintaining the temperature at −15° C. to −5° C. The solution was warmed to 15° C. to 25° C. and then stirred for 16 hr. Saturated aqueous NaHCO₃ (6 V) was added, and the mixture was extracted with DCM (4 V×2). The organic phase was washed with H₂O (10 V×5) and then concentrated to give the title compound (85%) as an oil.

Result: ¹H NMR (CDCl₃) δ 3.41 (s, 3H, C2′OCH₃), 3.84-4.09 (m, 8H, PCH₂, POCH₃), 4.26 (dd, 1H, C2′H), 5.23 (s, 1H, C4′H), 5.59 (d, 1H, C3′H), 5.90 (dd, 1H, C5-H), 6.50 (d, 1H, C1′H), 7.48-8.09 (m, 6H, C6-H, Ph-H), 8.11 (s, 1H, CONH). ES-MS m/z 369.10 (M−H).

### Example 7: Synthesizing dimethyl ((((2R,3S,4R,5R)-5-(2,4-dioxo-3,4-dihydropyrimidin-1 (2H)-yl)-3-hydroxy-4-methoxytetrahydrofuran-2-yl)oxy)methyl)phosphonate

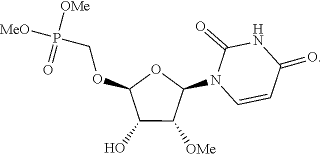 [SMILES: CO[C@@H]1[C@H](O)[C@@H](OCP(=O)(OC)OC)O[C@H]1n1ccc(=O)[nH]c1=O]

Method: [(2R,3S,4R,5R)-2-(dimethoxyphosphorylmethoxy)-5-(2,4-dioxopyrimidin-1-yl)-4-methoxy-tetrahydrofuran-3-yl]benzoate (1.0 eq) of Example 6 was dissolved in MeOH (10 V), then K₂CO₃ (1.5 eq) was added. The mixture was stirred for 6 hr at 20° C. to 30° C. and filtered. Then the pH was adjusted to about 4 to 5 by adding formic acid. The mixture was concentrated to a gummy solid and was purified by silica gel chromatography using a gradient of 60:1 to 30:1 CH₂Cl₂: MeOH to give the title compound (60%) as a solid.

Result: ¹H NMR (CD₃OD) δ 3.43 (s, 3H, C2′OCH₃), 3.82-3.86 (m, 6H, POCH₃), 4.00-4.10 (m, 3H, C3′H, PCH₂), 4.26 (dd, 1H, C2′H), 5.05 (s, 1H, C4′H), 5.81 (dd, 1H, C5-H), 6.32 (d, 1H, C1′H), 7.70 (d, 1H, C6-H). ES-MS m/z 367.20 (M+1).

### Example 8 Synthesizing 2-cyanoethyl ((2R,3S,4R,5R)-2-((dimethoxyphosphoryl)methoxy)-5-(2,4-dioxo-3,4-dihydropyrimidin-1 (2H)-yl)-4-methoxytetrahydrofuran-3-yl) diisopropylphosphoramidite (MeMOP)

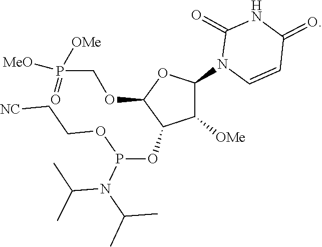 [SMILES: CO[C@@H]1[C@H](OP(OCCC#N)N(C(C)C)C(C)C)[C@@H](OCP(=O)(OC)OC)O[C@H]1n1ccc(=O)[nH]c1=O]

Method: Dimethyl ((((2R,3S,4R,5R)-5-(2,4-dioxo-3,4-dihydropyrimidin-1 (2H)-yl)-3-hydroxy-4-methoxytetrahydrofuran-2-yl)oxy)methyl)phosphonate (1.0 eq) of Example 7 was dissolved in DCM (3 V). A solution of DCM (10 V) and NMI (0.3 eq) was added to the mixture followed by dropwise adding of tetrazole (0.7 eq) and 3-((bis(diisopropylamino)phosphaneyl)oxy)propanenitrile (1.3 eq) while maintaining the temperature at 0° C. to 10° C. The solution was warmed to 15° C. to 25° C., stirred for 3 hr, and then a solution of 8% aqueous NaHCO₃ (8 V) was added. The layers were separated, and the organic phases were washed with 8% aqueous NaHCO₃ (5 V) followed by H₂O (5 V×4). The mixture was concentrated to about 1.5 V, and MTBE (15 V) was added. The mixture was filtered, and the filter cake was dried to give the title compound (64%) as a solid.

Result: ¹H NMR (CD₃CN) δ 1.22 (m, 12H, NCCH3), 2.73 (m, 2H, 2H, NCH), 3.39 (d, 3H, C2′OCH₃), 3.68-3.76 (m, 2H, CNCH2), 3.76-3.80 (m, 6H, POCH₃), 3.80-3.95 (m, 2H, OCH₂), 2.94-4.07 (m, 2H, PCH₂), 4.24 (dd, 1H, C2′H), 4.45 (dd, 1H, C3′H), 5.15 (d, 1H, C4′H), 5.74 (d, 1H, C5-H), 6.22 (d, 1H, C1′H), 7.60 (dd, 1H, C6-H), 9.09 (s, 1H, CONH). ES-MS (+ve mode)=567.20 (M+1).

1. A method of making a compound represented by a structure of: 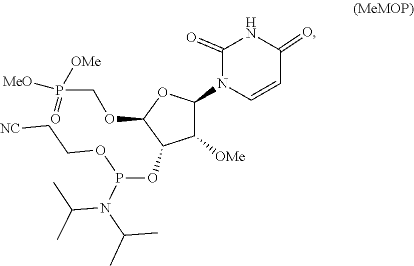 [SMILES: CO[C@@H]1[C@H](OP(OCCC#N)N(C(C)C)C(C)C)[C@@H](OCP(=O)(OC)OC)O[C@H]1n1ccc(=O)[nH]c1=O]
the method comprising the steps of:
oxidizing 1-((2R,3R,4R,5R)-4-hydroxy-5-(hydroxymethyl)-3-methoxytetrahydrofuran-2-yl)pyrimidine-2,4 (1H,3H)-dione to obtain (2S,3S,4R,5R)-5-(2,4-dioxo-3,4-dihydropyrimidin-1 (2H)-yl)-3-hydroxy-4-methoxytetrahydrofuran-2-carboxylic acid;
amidating and silylating (2S,3S,4R,5R)-5-(2,4-dioxo-3,4-dihydropyrimidin-1 (2H)-yl)-3-hydroxy-4-methoxytetrahydrofuran-2-carboxylic acid to obtain (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide;
adding an organometallic moiety to (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide to obtain 1-((2R,3R,4S,5S)-5-acetyl-4-((tert-butyldimethylsilyl)oxy)-3-methoxytetrahydrofuran-2-yl)pyrimidine-2,4 (1H,3H)-dione;
oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione to obtain (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate;
desilylating and benzoylating (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate to obtain (2R,3S,4R,5R)-2-acetoxy-5-(2,4-dioxo-3,4-dihydropyrimidin-1 (2H)-yl)-4-methoxytetrahydrofuran-3-yl benzoate;
hydrolyzing and alkylating (2R,3S,4R,5R)-2-acetoxy-5-(2,4-dioxo-3,4-dihydropyrimidin-1 (2H)-yl)-4-methoxytetrahydrofuran-3-yl benzoate to obtain [(2R,3S,4R,5R)-2-(dimethoxyphosphorylmethoxy)-5-(2,4-dioxopyrimidin-1-yl)-4-methoxy-tetrahydrofuran-3-yl]benzoate;
debenzoylating [(2R,3S,4R,5R)-2-(dimethoxyphosphorylmethoxy)-5-(2,4-dioxopyrimidin-1-yl)-4-methoxy-tetrahydrofuran-3-yl]benzoate to obtain dimethyl ((((2R,3S,4R,5R)-5-(2,4-dioxo-3,4-dihydropyrimidin-1 (2H)-yl)-3-hydroxy-4-methoxytetrahydrofuran-2-yl)oxy)methyl)phosphonate; and
phosphorylating dimethyl ((((2R,3S,4R,5R)-5-(2,4-dioxo-3,4-dihydropyrimidin-1 (2H)-yl)-3-hydroxy-4-methoxytetrahydrofuran-2-yl)oxy)methyl)phosphonate to obtain 2-cyanoethyl ((2R,3S,4R,5R)-2-((dimethoxyphosphoryl)methoxy)-5-(2,4-dioxo-3,4-dihydropyrimidin-1 (2H)-yl)-4-methoxytetrahydrofuran-3-yl) diisopropylphosphoramidite (MeMOP).

2. A method of making a compound represented by a structure of: 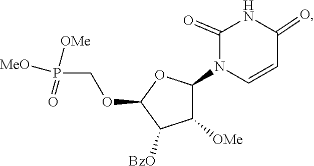
the method comprising the steps of:
oxidizing 1-((2R,3R,4R,5R)-4-hydroxy-5-(hydroxymethyl)-3-methoxytetrahydrofuran-2-yl)pyrimidine-2,4 (1H,3H)-dione to obtain (2S,3S,4R,5R)-5-(2,4-dioxo-3,4-dihydropyrimidin-1 (2H)-yl)-3-hydroxy-4-methoxytetrahydrofuran-2-carboxylic acid;
amidating and silylating (2S,3S,4R,5R)-5-(2,4-dioxo-3,4-dihydropyrimidin-1 (2H)-yl)-3-hydroxy-4-methoxytetrahydrofuran-2-carboxylic acid to obtain (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide;
adding an organometallic moiety to (2S,3S,4R,5R)-3-((tert-butyldimethylsilyl)oxy)-5-(2,4-dioxo-3,4-dihydropyrimidin-1 (2H)-yl)-N,4-dimethoxy-N-methyltetrahydrofuran-2-carboxamide to obtain 1-((2R,3R,4S,5S)-5-acetyl-4-((tert-butyldimethylsilyl)oxy)-3-methoxytetrahydrofuran-2-yl)pyrimidine-2,4 (1H,3H)-dione;
oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione to obtain (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate;
desilylating and benzoylating (2R,3S,4R,5R)-3-((tert-butyldimethylsilyl)oxy)-5-(2,4-dioxo-3,4-dihydropyrimidin-1 (2H)-yl)-4-methoxytetrahydrofuran-2-yl acetate to obtain (2R,3S,4R,5R)-2-acetoxy-5-(2,4-dioxo-3,4-dihydropyrimidin-1 (2H)-yl)-4-methoxytetrahydrofuran-3-yl benzoate; and
hydrolyzing and alkylating (2R,3S,4R,5R)-2-acetoxy-5-(2,4-dioxo-3,4-dihydropyrimidin-1 (2H)-yl)-4-methoxytetrahydrofuran-3-yl benzoate to obtain [(2R,3S,4R,5R)-2-(dimethoxyphosphorylmethoxy)-5-(2,4-dioxopyrimidin-1-yl)-4-methoxy-tetrahydrofuran-3-yl]benzoate.

3. The method of claim 1, wherein the step of oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione comprises a Baeyer Villiger reaction.

4. The method of claim 3, wherein the Baeyer Villiger reaction comprises the use of meta-chloroperoxybenzoic acid (mCPBA) or urea hydrogen peroxide (UHP).

5. The method of claim 2, wherein the step of oxidizing 1-((2R,3R,4S,5S)-5-acetyl-4-((tert-butyldimethylsilyl)oxy)-3-methoxytetrahydrofuran-2-yl)pyrimidine-2,4 (1H,3H)-dione comprises a Baeyer Villiger reaction.

6. The method of claim 5, wherein the Baeyer Villiger reaction comprises the use of meta-chloroperoxybenzoic acid (mCPBA) or urea hydrogen peroxide (UHP).
